(19) United States
(12) Patent Application Publication
(10) Pub. No.: US 2009/0023691 A1
(43) Pub. Date: Jan. 22, 2009

(54) DELAYING THE AGEING PROCESS AND DISORDERS CAUSED BY AGEING

(76) Inventor: Miso Sabovic, Ljubljana (SI)

Correspondence Address: BELL, BOYD & LLOYD, LLP, P.O. Box 1135, CHICAGO, IL, 60690, US

(21) Appl. No.: 11/910,952

(22) PCT Filed: Apr. 7, 2005
(86) PCT No.: PCT/EP05/03667

§ 371 Date: Jul. 28, 2008

(51) Int. Cl. A61K 31/625; A61K 31/351; A61K 31/404; A61P 9/00; A61P 35/00; A61P 25/00; A61K 31/41; A61K 31/355

(52) U.S. Cl. 514/161; 514/460; 514/419; 514/381; 514/458

(57) ABSTRACT

The present invention relates to the field of ageing, particularly to the prevention and/or treatment of ageing process and disorders caused by ageing and particularly to the prevention and/or treatment of atherosclerosis, neurodegenerative disorders and malignant diseases. A composition is provided, which comprises four or more active ingredients to reduce or suppress the ageing process or symptoms associated therewith.

The present invention relates to the field of ageing, particularly to the prevention and/or treatment of ageing process itself and the disorders caused by ageing, specifically to the prevention and/or treatment of atherosclerosis, neurodegenerative disorders and malignant diseases. A composition is disclosed, which comprises four or more active ingredients to reduce or suppress the ageing process or symptoms associated therewith.

The ageing process is characterized by a progressive, gradual decline of physiological functions of molecules, cells, tissues, organs and systems, and the impairment of individual functional (physical and cognitive) abilities. The mechanism(s) underlying the process of ageing are still poorly understood. Yet, it is widely believed that the ageing process could not be significantly modulated or slowed by pharmacological interventions. However, a growing body of scientific evidence has been published in the last decade, which directly or indirectly suggests that the ageing process could be modulated. Such efforts deal, however, preliminary the treatment of the skin with various cosmetics to decelerate the natural ageing process of the skin.

Currently known compositions for the treatment or prevention of ageing of the skin relate for example to a herbal anti-ageing agent with elastase inhibiting action and which is capable to maintain the tautness and elasticity of the skin and maintaining a youthful state of the skin (c.f. EP 0 919 223).

WO 2004060288 discloses topical compositions with an active ingredient derived from a plant or plant material for improving the appearance of skin, especially by alleviating skin irritation, wherein the topical compositions have at least one natural plant extract that inhibits COX-2 enzyme, NGF protein and/or TNF-alpha protein activity. In the FR 2837702 a composition particularly for the preparation of a night-cream is disclosed. The cream exhibits antioxidant effects and may be used for the prevention and or treatment of symptoms of ageing. Three herbal components are contained in the cream, which provide the anti ageing effect envisaged.

In US 2002197289 a cosmetic composition is disclosed, which contains fibers and at least one anti ageing active agent such as a vitamin.

Surprisingly, no or less efforts seem to have been made to combine findings from different fields of medicine in order to try to offer a scientifically-based pharmacological treatment with an achievable aim: not to prolong life, but primarily to delay and slow the functional decline associated with ageing not only of the skin. Three main mechanisms are regarded as responsible for particular “whole body” ageing processes, the vascular system ageing, oxidative stress, and chronic inflammations.

In the vascular ageing preliminary the large arteries, arterioles and the microcirculation are affected by degenerative ageing processes. In contrast to atherosclerosis, which merely affects large arteries, vascular ageing affects all parts of the arterial tree (large arteries, arterioles and microcirculation). The best marker of vascular ageing is the intima-media thickness (IMT) of the carotid arteries, which increases with age. Despite the common opinion that IMT is the precursor of atherosclerosis, it is, indeed, a marker of vascular ageing. For example, in mice resistant to atherosclerosis, IMT increases with ageing. In humans, the “aged” vessels are very prone to the development of atherosclerosis. It may be assumed that the basic mechanism underlying vascular ageing is the gradual over-expression and over-activity of angiotensin (Ang) II receptors (type AT1) in the vessel wall. This phenomenon, which is strongly individualized, could be facilitated by classic risk factors for atherosclerosis, and other factors as well, but, importantly, could also be slowed or even reversed by available pharmacological interventions.

During the ageing process the vascular wall, as well as parenchymal tissues, are exposed to gradually elevated oxidative stress with subsequent increased production of oxygen free radicals and consequent oxidative damage of lipids, proteins and DNA. An increased oxidative stress is the consequence of both, an increased production of free oxygen radicals and their decreased removal. Both processes are impaired by ageing, producing increased oxidative stress. The main source of oxidative stress in the vessel wall is NAD(P)H oxidase, the synthesis of which is intensified by Angiotensin II. In parenchymal organs other enzymes such as primarily the mitochondrial chain, xanthin oxidase and myeloperoxidase, are sources of free radicals. Oxidative stress increases progressively with ageing.

It is well known that ageing is also associated with fibrosis (accumulation of extra cellular matrix) affecting several organs and tissues. In contrast to the general belief that this is a degenerative process, it may be assumed that it is, in fact, an inflammatory one (there are similarities with the old and new definitions of atherosclerosis, which is now defined as chronic inflammatory disease, and not as a chronic degenerative disease as previously). The ageing-related inflammation process is characterized by a proliferative phase of inflammation (extra vascular matrix production and remodelling), rather that an exudative phase. Consequently, the fibrotic process occurs in parenchymal tissue, the central nervous system and the arterial wall. This results in a progressive damage of the affected organs. It is characteristic that during ageing the balance between Th1 and Th2 T-lymphocytes and the ratio between CD8 and CD4 surface T-cell receptors gradually changes toward the direction of higher production of pro-inflammatory cytokines and production of a low grade inflammation state.

Up to now, preliminary the use of natural compounds for the deceleration of the ageing process is known.

US-2004156881 discloses for example the use of different lignans for the production of foods with anti-inflammatory and/or anti-ageing properties.

In US-2004156950 different food products with anti-inflammatory and/or anti-ageing properties are specified. Active compounds are isoxanthohumol and/or xanthohumol, which are isolated from hop.

NZ 526350 relates to an anti-ageing composition, which includes metal salt(s), amino acid(s), an amino alcohol and a vitamin B complex. Additional components include L-Taurine, inositol, choline, heseperidine, an oligopeptide, resveratrol, folic acid, biotin, vitamin D, vitamin A, ginko biloba extract, lycopene, proanthocyanidin, black seed extract, bromelain, lipase, catalese, colloidal silica, iodine and an immuno-stimulant.

The regimen of the prior art did not, so far, provide a sufficient treatment of ageing. Thus, the problem of the present invention resides in the provision of a treatment regimen, which includes several active ingredients, wherein each ingredient bestows protection against one or more of the effects associated with the mechanisms identified as the reasons of the ageing processes of the vascular system and organs.

This objective has been achieved by the use of the present composition, which comprises a mixture of at least four constituents. Each of said constituents exhibits the capacity to achieve the aforementioned goal, namely to be included in a composition suitable for the prevention and/or treatment of ageing process and the disorders related to or caused by the natural ageing processes and particularly the prevention and/or treatment of atherosclerosis, neurodegenerative disorders and malignant diseases. The present invention pertains to the use of a statine, a compound suppressing angiotensin production or activity, an anti-inflammatory agent and at least one antioxidant.

In combination said constituents show surprisingly synergistic effects towards the prevention and/or treatment of disorders related to or caused by the natural ageing processes, in that the ageing processes are delayed or slowed. They also have been proven to be suitable for the prevention and/or treatment of atherosclerosis, neurodegenerative disorders and malignant diseases. Without wishing to be bound by any theory, it is presently assumed that the synergistic effects caused by the use of the different combined constituents reside in that on one hand the statine and likewise the compound suppressing angiotensin production or activity affect all three main mechanisms of aging (vascular aging, oxidative stress and anti-inflammatory action). On the other hand, the antioxidant counteracts oxidative stress, whereas the anti-inflammatory compound partially offsets tissue irritations. Depending on the choice of the particular compounds, they may also exhibit a protection against other ageing processes than specified above. Thus, the particular effect of the present composition resides in the combination of a compound, which prevents the generation of free radicals, a compound, which intercepts already generated free radicals, and compounds suppressing inflammations and ageing processes in some organs, particularly in the vascular system. Since vascular aging, oxidative stress and anti-inflammatory action are involved in several diseases (such as atherosclerosis, neurodegenerative diseases, etc.), the particular combination of the present compounds has important protective roles against said diseases as well.

According to a first embodiment of the present invention, the use of a composition for the prevention and/or treatment of a disorder caused by aging and the prevention and/or treatment of atherosclerosis, neurodegenerative disorders and malignant diseases is envisaged. Said composition comprises as active ingredients a statine, a compound suppressing angiotensin production, an anti-inflammatory agent and at least one antioxidant.

These constituents may, where desired, be present in form of a pharmaceutically acceptable salt, which may be prepared as alkaline metal or alkaline earth salts, such as sodium, potassium or calcium salts. Organic salts and esters are also suitable for use with this invention. The compositions as used in the present invention will, depending on the rout of administration contain other ingredients, such as e.g. excipients, surfactants, antioxidants, flavoring and coloring agents, fillers, volatile oils, buffering agents, dispersants, surfactants, antioxidants, flavoring agents, bulking agents, propellants and preservatives, etc. for the different formulations.

The statins are capable to affect all three main mechanisms of ageing. In addition, they have well-known anti-atherosclerotic effects and exhibit an anti-osteoporotic action. Since they also reveal a strong anti-inflammatory action, they are associated in the protection against numerous diseases. Any representative of the statins could be used. However, statins with definitely proven safety are preferred.

Like the statins, compounds suppressing angiotensin production or activity effectively retard all three main mechanisms of aging. Since the over-expression of local renin-angiotensin (RAS) is involved in several diseases, such as atherosclerosis, neurodegenerative diseases, etc., a combination of these compounds have important protective roles against said diseases as well.

According to an embodiment of the present invention said neurodegenerative disorders are selected from the group consisting of Alzheimer's disease, Parkinson's disease, amyotrophic lateral sclerosis (ALS), hereditary cerebral hemorrhage with amyloidosis, cerebral amyloid angiopathy, prion-mediated diseases, inclusion body myositis, stroke, multiple sclerosis, Down's Syndrome and Huntington's disease.

According to another embodiment, the malignant diseases are caused by a tumour, such as e.g. carcinoma, melanoma and sarcoma.

The compositions according to the present invention may be administered, orally, intracavitarily, intranasally, intraanally, intravaginally, intrauterally, intracranially, pulmonarily, intrarenally, intranodularly, intraarticularly, intraotically, intralymphatically, transdermally, intrabucally, intravenously, subcutaneously, intramuscularly, intratumorously, intraglandularly, intraocularly, intracranial, into an organ, intravascularly, intrathecally, by implantation, by inhalation, intradermally, intrapulmonarily, by slow release, by sustained release etc.

The administration of the composition may be conducted with an oral formulation having a liquid carrier such as solutions, suspensions, and oil-in-water and water-in-oil emulsions, and/or may be administered as a powder, dragees, tablets, capsules, sprays, aerosols, solutions, suspensions and emulsions. The composition may be administered once or several times a day.

When the formulation is injectable, the carrier may be selected from aqueous and alcoholic solutions and suspensions, oily solutions and suspensions and oil-in-water and water-in-oil emulsions, among others. When applied as a rectal formulation, it may be in the form of a suppository, when in the form of a transdermal formulation, the carrier may be selected among aqueous and alcoholic solutions, oily solutions and suspensions and oil-in-water and water-in-oil emulsions, etc.

The transdermal formulation, may be aniontophoretic transdermal formulation, wherein the carrier is selected aqueous and alcoholic solutions, oily solutions and suspensions and oil-in-water and water-in-oil emulsions, and the formulation may also contain a transdermal transport promoting agent, of which many are known in the art. Also suitable for prolonged administration are implantable capsules or cartridges containing the formulation. In this case, the carrier may also be selected from aqueous and alcoholic solutions and suspensions, oily solutions and suspensions and oil in-water and water-in-oil emulsions, be a hydrophobic carrier, such as lipid vesicles or particles, e. g. liposomes made of N-(1-[ 2,3-dioleoxyloxi] propyl)-N, N,N- trimethyl-ammonium methylsulfate, and/or other lipids, and microcrystals.

For pulmonary applications, the formulation is preferably a respirable or inhalable formulation, e. g. in the form of an aerosol. For prolonged exposure the composition may be delivered through a localized implant, suppository, sublingual formulation, etc., all of which are known in the art.

Formulations suitable for respiratory, nasal, intrapulmonary, and inhalation administration are preferred, as are topical, oral and parenteral formulations. All methods of preparation include the step of bringing the active compound into association with a carrier which constitutes one or more accessory ingredients. In general, the formulations are prepared by uniformly and intimately bringing the constituents into association with a liquid carrier, a finely divided solid carrier, or both, and then, if necessary, shaping the product into desired formulations.

Compositions suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion.

Compositions suitable for parenteral administration comprise sterile aqueous and non-aqueous injection solutions of the active compound, which preparations are preferably isotonic with the blood of the intended recipient. These preparations may contain anti-oxidants, buffers, bacteriostats and solutes which render the compositions isotonic with the blood of the intended recipient. Aqueous and non-aqueous sterile suspensions may include suspending agents and thickening agents. The compositions may be presented in unit-dose or multi-dose containers, for example sealed ampoules and vials, and may be stored in a freeze-dried or lyophilized condition requiring only the addition of the sterile liquid carrier, for example, saline or water-for-injection immediately prior to use.

Nasal and instillable formulations comprise purified aqueous solutions of the active compound with preservative agents and isotonic agents. Such formulations are preferably adjusted to a pH and isotonic state compatible with the nasal mucous membranes.

Ophthalmic formulations are prepared by a similar method to the nasal spray, except that the pH and isotonic factors are preferably adjusted to match that of the eye. Otical formulations are generally prepared in viscous carriers, such as oils and the like, as is known in the art, so that they may be easily administered into the ear without spilling.

The composition disclosed herein may be administered into the respiratory system either by inhalation, respiration, nasal administration or intrapulmonary instillation (into the lungs) of a subject by any suitable means, and are preferably administered by generating an aerosol or spray comprised of powdered or liquid nasal, intrapulmonary, respirable or inhalable particles. The formulation may comprise respirable or inhalable liquid or solid particles of the active compound that, in accordance with the present invention, include respirable or inhalable particles of a size sufficiently small to pass through the mouth and larynx upon inhalation and continue into the bronchi and alveoli of the lungs. In general, particles ranging from about 0.05, about 0.1, about 0.5, about 1, about 2 to about 4, about 6, about 8, about 10 um in diameter. More particularly, about 0.5 to less than about 5 μm in diameter, are respirable or inhalable. For nasal administration or intrapulmonary instillation, a particle size in the range of about 8, about 10, about 20, about 25 to about 35, about 50, about 100, about 150, about 250, about 500 μm (diameter) is preferred to ensure retention in the nasal cavity or for instillation and direct deposition into the lung. Liquid pharmaceutical compositions of active compound for producing an aerosol may be prepared by combining the active compound with a stable vehicle, such as sterile pyrogen free water. A solid particulate composition comprised of the composition may optionally contain a dispersant that serves to facilitate the formation of an aerosol. A suitable dispersant is lactose, which may be blended with the composition in any suitable ratio, e.g. a 1 to 1 ratio by weight.

Optional additives include preservatives if the composition is not prepared sterile, for example, methylhydroxybenzoate, anti-oxidants, flavoring agents, volatile oils, buffering agents and surfactants. Aerosols of solid particles comprising the active compound may likewise be produced with any sold particulate medicament aerosol generator. Aerosol generators for administering solid particulate medicaments to a subject product particles which are respirable, as explained above, and generate a volume of aerosol containing a predetermined metered dose of a medicament at a rate suitable for administration.

The composition may be delivered with any delivery device that generates liquid or solid particulate aerosols, such as aerosol or spray generators. These devices produce respirable particles, as explained above, and generate a volume of aerosol or spray containing a predetermined metered dose of a medicament at a rate suitable for human or animal administration. One illustrative type of solid particulate aerosol or spray generator is an insufflator, which are suitable for administration of finely comminuted powders. In the insufflator, the powder, e. g. a metered dose of the composition effective to carry out the treatments described herein, is contained in a capsule or a cartridge. These capsules or cartridges are typically made of gelatine, foil or plastic, and may be pierced or opened in situ, and the powder delivered by air drawn through the device upon inhalation or by means of a manually-operated pump.

According to an embodiment, the statin is rather selected from the group consisting of lova-, simva-, prava- and fluvastatin. Preferably fluvastatin is used, which has a pronounced effect on the elevation of HDL cholesterol (which itself has important anti-oxidative properties). Furthermore, fluvastatin is a lipid soluble molecule, which simply passes cell membrane. As other statins, fluvastatin exhibits pleotrophic effects. Fluvastatin has also an important systemic anti-inflammatory effect and decreases the expression of Angiotensin II receptors in the vessel wall. Thus, fluvastatin possesses anti-inflammatory and anti-oxidative properties, and directly hinders the vessel aging. Fluvastatin exhibits additionally a safety profile above average and has been in clinical use for a long period in comparison to other statins.

The compound, capable to suppress the angiotensin production or activity, is preferably selected from the group consisting of an angiotensin-converting enzyme (ACE) inhibitors and angiotensin II receptor blockers. Since basic mechanisms of ageing rely on increased oxidative stress and overexpression of the RAS system, the use of angiotensin receptor blockers (ARBs) or angiotensin-converting enzyme (ACE) inhibitors have proven beneficial effects in the present composition. In contrast to antioxidants, such as vitamins, ARBs and ACE inhibitors reduce the production of free radicals and do not scavenge them like the antioxidant vitamins. Different representatives of ARBs or ACE inhibitors, which are well known to the skilled person, may be chosen for the present composition.

According to another embodiment of the present invention, the ARB is preferably selected from the group consisting of valsartan, losartan, irbisartan, etc., and ACE inhibitors from the group consisting of perindopril, trandolapril, ramipril, etc. Said lipophilic agents exhibit a higher effectiveness than other ARBs and ACE inhibitors, due to an improved cell uptake and bioavailability. Said ARBs and ACE inhibitors are also non-allergic, chemically stable and bioactive.

A preferred ARB is the compound valsartan, which reveals a high specificity for the inhibition of Angiotensin II AT1 receptors in the arterial wall, and in parenchyma organs (heart, kidney). Additionally, valsartan has an anti-inflammatory capacity. Thus, valsartan is capable to affect directly and indirectly all three main mechanisms of the aging process.

According to an embodiment, said anti-inflammatory agent is acetylsalicylic acid, which exhibits a strong systemic anti-inflammatory action, decreases oxidative stress and might also be protective against colon cancer.

According to another preferred embodiment said at least one antioxidant is a vitamin, which is preferably selected from the group consisting of vitamin A, vitamin C and vitamin E. Likewise, derivatives of said vitamin(s) may be included in the present composition. More preferably a combination of the lipophilic vitamin E and hydrophilic vitamin C is used, since said combination is capable to effectively prevent both “vascular” and “parenchymal” oxidative stress due to the different solubility profiles of said compounds.

Vitamin A or retinoids, either naturally occurring or synthetic, are defined by their ability to bind nuclear retinoid receptors of the steroid/thyroid superfamily. Their protean but key function in physiology is control of cellular proliferation and differentiation. In the treatment of photo ageing, numerous large-scale, double-blind, placebo-controlled trials have already shown the efficacy of topical retinoin. Topical retinoids, namely retinoin, prevent and repair clinical features of ageing processes of the skin. However, as retinoin remains a prescription drug, over-the-counter products mainly utilize the vitamin A derivatives retinol and retinyl palmitate. These forms are not biologically active until enzymatic conversion to the principal active metabolite, retinoic acid and are also useful in the prevention and/or treatment of aging processes not only confined to the skin.

The hydrophilic vitamin C or L-ascorbic acid, the biologically active form of vitamin C, has well-established roles in the human body as an antioxidant and as a cofactor for collagen synthesis. Ascorbate participates in the hydroxylation of procollagen, and studies show that it may also stimulate collagen synthesis directly by activating its transcription and stabilizing procollagen mRNA. The physiological mechanisms affected by vitamin C are likely to include the increased production of collagen, as well as the decreased production of matrix metalloproteinase, an enzyme that enhances dermal collagen degradation. Ascorbic acid exhibits also strong anti-inflammatory effects. Further, its role as an antioxidant gives ascorbate similar photo protecting properties as vitamin E by neutralizing UVB-generated free radicals. Beyond its well-known antioxidant effects vitamin C may also have cytoprotective effects. As an antioxidant, ascorbic acid is the body's main water-soluble, non-enzymatic scavenger of free radicals, enabling it to function efficiently in aqueous compartments. Additionally, ascorbic acid also helps to regenerate the oxidized forms of α-tocopherol. Stable and hydrophobic solutions of ascorbic acid consist of the molecule in its non-ionized form at a low pH.

Vitamin E, or α-tocopherol, is a lipid-soluble antioxidant, which plays key roles in protecting cellular membranes from lipid peroxidation by free radicals. These free radicals contribute significantly to the environmental or extrinsic ageing. Although the human tissues possesses various intrinsic defence systems which help to mitigate these types of oxidative damage, both excessive and chronic exposure to free radicals can deplete the body's defence, α-tocopherol modulates this damage by scavenging free radicals and lipid peroxyl radicals. Vitamin E is distributed in a gradient fashion in the stratum corneum of healthy skin, with the highest levels in the deepest layers and the lowest levels closest to the surface. Vitamin E exhibits further mainly in the parenchymal organs and less in the vascular wall a strong antioxidant activity. Vitamin E is available most commonly as α-tocopherol or tocopherol acetate. The protective effects of vitamin E against photo ageing have been already demonstrated in various animals and in vitro skin models and suggest thereby also the use for the prevention and/or treatment of a disorder caused by ageing processes or for the prevention and/or treatment of atherosclerosis, neurodegenerative disorders and malignant diseases. The advantage of this vitamin resides in its lipophilic property which allows an effective inhibition (scavenge) of oxygen free radicals produced in the cell (mainly produced in the respiratory chain).

The composition is administered in an amount to be effective for the intended application and the subject to be treated. To this end, the dosage of the composition and or its constituents may vary depending on age, weight, and condition of the subject. In general, the active agent is preferably administered at a concentration that will afford effective results without causing any harmful or deleterious side effects, and may be administered either as a single unit dose, or if desired in convenient subunits administered at suitable times throughout the day.

According to a preferred embodiment, the statin will be contained in the composition in an amount of about 0.12 to 4.0 mg per single (daily) dose. These amounts per single dose are notedly lower than the recommended daily doses of statines, which are for example in the case of fluvostatine 12-16 mg (40-80mg) per day and, thus, do not lead to any side-effects. Preferably, the statin is contained in an amount of about 0.12 to 4.0 mg and more preferably in an amount of about 1.8 to 3.2 mg per single (daily) dose.

According to another embodiment, the compound suppressing angiotensin production or activity is contained in the composition in an amount of about 0.16 to 5.5 mg per single (daily) dose. Also these amounts per single dose are lower than the recommended daily doses of compounds having the capability to suppress the angiotensin production, which are for example in the case of valsartan 16-24 mg per day (80-160 mg), and do not lead to any side-effects. Preferably, the compound suppressing angiotensin production or activity is contained in an amount of about 2.0 to 5.5 mg and more preferably in an amount of about 2.4 to 4.8 mg per single (daily) dose.

According to still another embodiment, said anti-inflammatory agent is contained in the composition in an amount of about 20 to 200 mg, preferably in an amount of about 20 to 100 mg and more preferably in an amount of about 20 to 50 mg. A single (daily) dose may comprise for example 50 mg of acetylsalicylic acid per single (daily) dose. From acetylsalicylic acid it is already well known, that the low concentrations as envisaged do not exhibit any side-effects.

According to an embodiment, said at least one antioxidant is contained in the composition in an amount of about 100 to 1000 mg, wherein only vitamin C may be added in amounts exceeding 200 mg. Said at least one antioxidant is preferably added in an amount of about 300 to 1000 mg and more preferably in an amount of about 500 to 700 mg. A single (daily) dose may comprise for example 500 mg of vitamin C and 200 IU of vitamin E (1 IU of vitamin E is defined as the specific biological activity of 0.671 milligram of d-alpha-tocopherol or 1.0 milligram of dl-alpha-tocopherol acetate). Also from the antioxidants it is already well known, that the low concentrations as envisaged do not exhibit any detrimental side-effects.

The present invention also provides for a kit comprising the composition and a delivery device. The compositions may conveniently be presented in single or multiple unit dosage forms as well as in bulk, and may be prepared by any of the methods which are well known in the art of pharmacy.

The following examples illustrate the invention without limiting it thereto.

### EXAMPLES

1. Preparation of about 1.3 g tablet A:

| | | |
|---|---|---|
| 3.2 | mg | fluvostatin, |
| 4.8 | mg | valsartan, |
| 50 | mg | acetylsalicylic acid, |
| 500 | mg | vitamin C, |
| 134 | mg | vitamin E, |
| 408 | mg | lactose monohydrate, |
| 200 | mg | corn starch, and |
| 0.015 | mg | magnesium-stearate. |

Followed by blending to achieve a homogenous consistence and pressing.

1. Preparation of about 650 mg tablet B:

| | | |
|---|---|---|
| 1.8 | mg | fluvostatin, |
| 2.4 | mg | valsartan, |
| 50 | mg | acetylsalicylic acid, |
| 250 | mg | vitamin C, |
| 67 | mg | vitamin E, |
| 186.8 | mg | lactose monohydrate, |
| 92 | mg | corn starch, and |
| 0.015 | mg | magnesium-stearate. |

Followed by blending to achieve a homogenous consistence and pressing.

3. Assay for the influence of the active ingredients on cell growth and number of juvenile cells

Pre-characterized ovine aortic tissue cells, deposited with DSM-Deutsche Sammlung von Mikroorganismen and the Accession No. DSM ACC349, have been used to study the influence of the respective active ingredients.

The tissue cells were grown in 96-well microtitreplates, containing 150 μl/well 90% RPMI/10% FBS medium, preheated at 37° C. and 5% CO₂ to a density of≧1000 cells/well up to 1500 cells/well, determined by means of a Thoma chamber under a light microscope. 90% RPMI/10% FBS medium (cf. DSMZ catalogue, Human and Animal Cell Lines, 1999) represents also the medium for cell growth and was changed daily.

The influence of the active ingredients of ointments A and B have been tested by preparing solutions containing 15 mg valsartan, 30 mg vitamin A, 30 mg vitamin C, 30 mg vitamin E, 30 mg 2-hydroxy-4-methoxybenzophenone in 10 ml 90% RPMI/10% FBS medium (solution A) and 25 mg valsartan, 50 mg vitamin A, 50 mg vitamin C, 50 mg vitamin E, 25 mg 2-hydroxy-4-methoxybenzophenone and 25 mg 1-phenyl-3-(4′-isopropylphenyl)propane-1,3-dione in 10 ml RPMI/FBS medium (solution B). Dissolving of the components in 90% RPMI/10% FBS medium was promoted by using of an ultrasonic-bath at ambient temperature. As negative control 90% RPMI/10% FBS medium (solution C) has been used.

Different amounts (0, 1, 5, 10, 25 μl) of each solution (A, B or C) were added to each well. Subsequently, amounts of 25, 24, 20, 15 and 0 μl of 90% RPMI/10% FBS medium were added to each well to maintain a constant total volume. The growth behaviour and the amount of juvenile cells was checked after 48 hours. Therefore, a 50 μl sample was removed from each well. The cell number and the number of juvenile cells was determined by means of a Thoma chamber and divided by the initial cell amount of the respective cells. The cell number and number of juvenile cells in wells treated with 0 or 1 μl of solutions A, B or C were regarded as basic values and compared to that of wells treated with 5, 10 or 25 μl of solution A, B or C, respectively. In comparison to the basic values, the addition of 5 μl solution A, B or C did not result in a detectable effect, since the values were within a 0.025 error range. The ratio of the cell number per initial cell number divided by the basic value obtained by addition of 10 and 25 μl of each solution are shown in table 1.

TABLE 1

| | Solution A | Solution B | Solution C |
|---|---|---|---|
| 10 μl | 1.04 | 1.05 | 1.02 |
| 25 μl | 1.09 | 1.10 | 0.99 |

The ratio of the number of juvenile cells per initial cell number divided by the basic value obtained by addition of 10 and 25 μl of each solution are shown in table 2.

TABLE 2

| | Solution A | Solution B | Solution C |
|---|---|---|---|
| 10 μl | 1.03 | 1.05 | 0.99 |
| 25 μl | 1.07 | 1.11 | 1.01 |

These values indicate that amounts of 10 and 25 μl of solution A and B have both a positive effect towards cell growth and cell reproduction, in that increased numbers of juvenile cells were detected.

1. A method for producing a composition, comprising the steps of using
a statin,
a compound suppressing angiotensin production or activity,
an anti-inflammatory agent; and
at least one antioxidant to produce the composition.

2. A method for treating a disease selected from the group consisting of atherosclerosis, neurodegenerative disorders, and malignant diseases, comprising the step of administering to a patient having the disease a composition, comprising
a statin,
a compound suppressing angiotensin production or activity,
an anti-inflammatory agent; and
at least one antioxidant.

3. The method according to claim 2, wherein the neurodegenerative disorders is selected from the group consisting of Alzheimer's disease, Parkinson's disease, amyotrophic lateral sclerosis (ALS), hereditary cerebral hemorrhage with amyloidosis, cerebral amyloid angiopathy, prion-mediated diseases, inclusion body myositis, stroke, multiple sclerosis, Down's Syndrome and Huntington's disease.

4. The method according to claim 2, wherein the malignant diseases is caused by a tumour.

5. The method according to claim 1, wherein the composition is in a form selected from the group consisting of a tablet, capsule and a liquid form.

6. The method according to claim 1, wherein the statin is selected from the group consisting of lova-, simva-, prava- and fluvastatin.

7. The method according to claim 1, wherein the compound suppressing angiotensin production or activity is selected from the group consisting of an angiotensin-converting enzyme inhibitors and angiotensin II receptor blockers.

8. The method according to claim 7, wherein the angiotensin II receptor blockers or angiotensin-converting enzyme inhibitors is selected from the group consisting of valsartan, losartan, irbisartan, trandolapril, perindopril and ramipril.

9. The method according to claim 1, wherein the anti-inflammatory agent is acetylsalicylic acid.

10. The method according to claim 1, wherein the at least one antioxidant is a vitamin.

11. The method according to claim 10, wherein the vitamin is selected from the group consisting of vitamin A, vitamin C and vitamin E.

12. The method according to claim 1, wherein the statin is contained in an amount of 0.12 to 4.0 mg per single dose.

13. The method according to claim 1, wherein the compound suppressing angiotensin production or activity comprises 0.16 to 5.5 mg per single dose.

14. The method according to claim 1, wherein the anti-inflammatory agent is present is in an amount of 20 to 200 mg.

15. The method according to claim 1, wherein the one antioxidant is present in an amount of 100 to 1000 mg.

16. A method for treating disorders caused by ageing comprising the steps of administering to a patient having same a composition, comprising a statin, a compound suppressing angiotensin production or activity, an anti-inflammatory agent; and at least one antioxidant.

17. A method for preventing antherosclerosis, neurodegenerative disorders and malignant disorders comprising the step of administering to a patient at risk of same a composition, comprising a statin, a compound suppressing angiotensin production or activity, an anti-inflammatory agent; and at least one antioxidant.

18. The method according to claim 17, wherein the neurodegenerative disorders is selected from the group consisting of Alzheimer's disease, Parkinson's disease, amyotrophic lateral sclerosis (ALS), hereditary cerebral hemorrhage with amyloidosis, cerebral amyloid angiopathy, prion-mediated diseases, inclusion body myositis, stroke, multiple sclerosis, Down's Syndrome and Huntington's disease.

19. The method according to claim 17, wherein the malignant diseases are caused by a tumour.

20. The method according to claim 2, wherein the composition is in a form selected from the group consisting of a tablet, capsule and a liquid form.

21. The method according to claim 2, wherein the statin is selected from the group consisting of lova-, simva-, prava- and fluvastatin.

22. A method for preventing disorders caused by ageing comprising the steps of administering to a patient at risk of same a composition, comprising a statin, a compound suppressing angiotensin production or activity, an anti-inflammatory agent; and at least one antioxidant.

23. The method according to claim 16, wherein the neurodegenerative disorders is selected from the group consisting of Alzheimer's disease, Parkinson's disease, amyotrophic lateral sclerosis (ALS), hereditary cerebral hemorrhage with amyloidosis, cerebral amyloid angiopathy, prion-mediated diseases, inclusion body myositis, stroke, multiple sclerosis, Down's Syndrome and Huntington's disease.

24. The method according to claim 16, wherein the malignant diseases are caused by a tumour.

25. The method according to claim 2, wherein the compound suppressing angiotensin production or activity is selected from the group consisting of angiotensin-converting enzyme inhibitors and angiotensin II receptor blockers.

26. The method according to claim 16, wherein the compound suppressing angiotensin production or activity is selected from the group consisting of angiotensin-converting enzyme inhibitors and angiotensin II receptor blockers.

27. The method according to claim 17, wherein the compound suppressing angiotensin production or activity is selected from the group consisting of angiotensin-converting enzyme inhibitors and angiotensin II receptor blockers.

28. The method according to claim 25, wherein the angiotensin II receptor blockers or angiotensin-converting enzyme inhibitors are selected from the group consisting of valsartan, losartan, irbisartan, trandolapril, perindopril and ramipril.

29. The method according to claim 26, wherein the angiotensin II receptor blockers or angiotensin-converting enzyme inhibitors are selected from the group consisting of valsartan, losartan, irbisartan, trandolapril, perindopril and ramipril.

30. The method according to claim 27, wherein the angiotensin II receptor blockers or angiotensin-converting enzyme inhibitors are selected from the group consisting of valsartan, losartan, irbisartan, trandolapril, perindopril and ramipril.

31. The method according to claim 2, wherein the anti-inflammatory agent is acetylsalicylic acid.

32. The method according to claim 16, wherein the anti-inflammatory agent is acetylsalicylic acid.

33. The method according to claim 17, wherein the anti-inflammatory agent is acetylsalicylic acid.

34. The method according to claim 2, wherein the at least one antioxidant is a vitamin.

35. The method according to claim 16, wherein the at least one antioxidant is a vitamin.

36. The method according to claim 17, wherein the at least one antioxidant is a vitamin.

37. The method according to claim 34, wherein the vitamin is selected from the group consisting of vitamin A, vitamin C and vitamin E.

38. The method according to claim 35, wherein the vitamin is selected from the group consisting of vitamin A, vitamin C and vitamin E.

39. The method according to claim 36, wherein the vitamin is selected from the group consisting of vitamin A, vitamin C and vitamin E.

40. The method according to claim 2, wherein the statin comprises 0.12 to 4.0 mg per single dose.

41. The method according to claim 16, wherein the statin comprises 0.12 to 4.0 mg per single dose.

42. The method according to claim 17, wherein the statin comprises 0.12 to 4.0 mg per single dose.

43. The method according to claim 2, wherein the compound suppressing angiotensin production or activity comprises 0.16 to 5.5 mg per single dose.

44. The method according to claim 16, wherein the compound suppressing angiotensin production or activity comprises 0.16 to 5.5 mg per single dose.

45. The method according to claim 17, wherein the compound suppressing angiotensin production or activity comprises 0.16 to 5.5 mg per single dose.

46. The method according to claim 2, wherein the anti-inflammatory agent is present at a level of 20 to 200 mg.

47. The method according to claim 16, wherein the anti-inflammatory agent is present at a level of 20 to 200 mg.

48. The method according to claim 17, wherein the anti-inflammatory agent is present at a level of 20 to 200 mg.

49. The method according to claim 2, wherein said at least one antioxidant is present at a level of 100 to 1000 mg.

50. The method according to claim 16, wherein said at least one antioxidant is present at a level of 100 to 1000 mg.

51. The method according to claim 17, wherein said at least one antioxidant is present at a level of 100 to 1000 mg.
